(19) United States
(12) Patent Application Publication
(10) Pub. No.: US 2015/0079648 A1
(43) Pub. Date: Mar. 19, 2015

(54) Biological Stabilization for Fermentable Biomass

(71) Applicant: Jacob R. Borden, Lake Charles, LA (US)

(72) Inventor: Jacob R. Borden, Lake Charles, LA (US)

(21) Appl. No.: 14/491,972

(22) Filed: Sep. 19, 2014

Related U.S. Application Data
(60) Provisional application No. 61/880,168, filed on Sep. 19, 2013.

(51) Int. Cl. C12P 7/06; C12P 7/54; C12P 7/56

(52) U.S. Cl. CPC C12P 7/06; C12P 7/56; C12P 7/54
USPC 435/139; 435/140; 435/161; 435/255.2

(57) ABSTRACT

A stabilized biomass and a method of producing a stabilized biomass is disclosed. The biomass has active matter containing carbon atoms having an average oxidation state, inactive matter, a biological catalyst having a fermentation organism capable of converting the active matter into a renewable material, and water. The biomass has not been milled. The biomass is suitable for use in the production of renewable materials, such as ethanol.

### CROSS REFERENCE TO RELATED APPLICATION

This application claims priority to U.S. provisional patent application Ser. No. 61/880,168, filed Sep. 19, 2013 and entitled, “Biological Stabilization for Fermentable Biomass,” naming Jacob R. Borden, Ph.D., as the inventor.

### FIELD OF THE INVENTION

This disclosure relates generally to a biologically-stabilized biomass, in particular cereal grains such as barley, corn, oats, rice, rye or wheat and sugar crops such as sugar cane, sugar beets, or Jerusalem artichoke, that are useful as formation feedstock for producing renewable materials. The disclosure also relates to a method of producing the biologically-stabilized biomass. Specific applications can include a biomass treatment unit for producing the biologically-stabilized biomass.

### BACKGROUND OF THE INVENTION

Concern over climate change and greenhouse gas levels has led to the development of technologies that utilize natural cycles between fixed carbon and liberated carbon dioxide. Among the available technologies, considerable advances have been made in the fermentation of photosynthesized carbohydrates, e.g. cereal grain crops such as barley, corn, oats, rice, rye or wheat, and other crops such as sugarcane, Jerusalem artichoke, sugar beets to produce renewable materials such as biofuels and biochemicals.

Examples of such desirable renewable end products include biofuels and biochemicals, bio methane, biogas, amino acids, proteins, lipids, alcohols, esters, organic acids and the like.

However, even with recent advances in technology, such as the development of (a) genetically modified corn varieties with improved starch and overall bushel per acre yields and improved resistance to drought and pestilence, (b) agricultural implements with improved on-board nutrient analysis and real-time fertilizer application, and (c) genetically-modified biological catalysts that produce improved yield and productivity of renewable end-products, have improved resistance to fermentation-inhibiting compounds, and have improved metabolism of complex sugar mixtures including five-carbon and six-carbon sugars, there remains a need and a desire to improve the economic viability for conversion of renewable carbon sources to fuels by reducing losses of these renewable carbon sources due to undesirable spoilage during storage by native organisms that are always present on the surface of these crops.

This spoilage occurs between harvesting the crops and using them as biomass for production of renewable materials. Undesirable spoilage is defined as the conversion by native organisms always present on the surface of these crops, of metabolizable carbohydrates (such as monosaccharides, disaccharides, and oligosaccharides) into undesirable organic acids. This spoilage reduces the yield of desirable renewable end products (such as ethanol or other bio fuels or biochemicals) than can be produced by desirable fermentation with biological catalysts.

Additionally, economic viability for conversion of renewable carbon sources to fuels can be improved by reducing the time and energy required to convert these cereal grain crops and sugar crops into an economically desirable end-product.

As cereal crops (e.g., barley, corn, oats, rice, rye or wheat) reach maturity, kernel moisture content naturally decreases in equilibrium with the decreasing relative humidity of late summer and early fall. Depending on ambient conditions, field-drying results in moisture content of about 20%. But 20% is still enough moisture to support the growth of native organisms, so extended field-drying can lower the yield of readily-metabolized carbohydrates (typically monosaccharides, disaccharides, and oligosaccharides) in these grains into economically desirable end-products, because these carbohydrates are metabolized by the undesirable native microbes.

Currently, grains, sugarcane, Jerusalem artichoke and sugar beets must be force-dried to 15-16% moisture by weight within 48 hours of harvest if they are to be used as biomass to produce desirable renewable products. This level of water is low enough to prevent metabolism of carbohydrates such as monosaccharides, disaccharides, and oligosaccharides by the native microbes into organic acids.

This water removal is currently done using either of two methods, and both methods tend to reduce the levels of metabolizable carbohydrates such as monosaccharides, disaccharides, and oligosaccharides in these crops.

The first method uses high-temperature, low residence time dryers that effectively and rapidly drive off water, but the high temperatures convert a portion of the monosaccharides, disaccharides, and oligosaccharides into char or Maillard-type reaction products, neither of which can then be fermented by yeast.

The second method uses forced-draft fans without heat to drive off water. This method suffers from requiring a longer residence time, during which the native organisms can degrade the monosaccharides, disaccharides, and oligosaccharides into undesirable organic acids.

Both of these methods consume energy.

In addition, the biological catalysts used to produce desirable renewable end products require water to effect the conversion of metabolizable carbohydrates into desirable renewable end products, and therefore, water must be added back to the dried biomass in order for the conversion to take place.

Thus, a problem associated with methods of stabilization that precede the present disclosure is that they do not provide, in combination with the other features and advantages disclosed herein, a method that can stabilize and prevent undesirable spoilage of the metabolizable carbohydrates in cereal grains without the need for forced-air drying.

Another problem associated with methods and devices that precede the present disclosure is that they do not provide, in combination with the other features and advantages disclosed herein, a method that can reduce the time, energy and additional water needed to convert these cereal grains and sugar crops into an economically desirable end-product.

Yet another problem associated with methods and devices that precede the present disclosure is that they do not provide, in combination with the other features and advantages disclosed herein, a method that can prevent undesirable spoilage by native organisms of the metabolizable carbohydrates in grains and sugar crops without substantial processing to renewable products within about 48 hours of harvest.

There is a demand, therefore, to overcome the foregoing problems while at the same time providing a method that will simultaneously prevent undesirable spoilage of the readily-metabolized carbohydrates in these cereal grains and sugar crops and reduce the time and energy needed to convert these cereal grains and sugar crops into economically desirable end-products.

### SUMMARY OF THE INVENTION

In a preferred embodiment, the disclosure provides biologically-stabilized biomass suitable for use in the production of renewable materials, the biologically-stabilized biomass comprising an inoculating biological catalyst, water, active matter and inactive matter, wherein the biomass has not been force-dried.

In another preferred embodiment, the inoculating biological catalyst is a fermentation organism capable of consuming at least a portion of the active matter in the biomass and converting the active matter into a renewable material. Examples of such a biological catalyst include cyanobacteria, fungus, algae, yeast, ethanologenic yeast, diatom, phytoplankton or genetically modified organism.

In another preferred embodiment, the disclosure comprises a method of stabilization of otherwise labile carbohydrates by treating the cereal grain or sugar crop within 48 hours of harvest with a biological catalyst, e.g. cyanobacteria, fungus, algae, yeast, ethanologenic yeast, diatom, phytoplankton or genetically modified organism, which produces an intended renewable end-product. Importantly, the cereal grain or sugar crop is not force-dried at any point.

In still another preferred embodiment, the cereal kernel or sugar crop hull itself remains whole, or intact when the crop is treated with a biological catalyst. The intact kernel or hull provides a semi-permeable barrier whereby the biological catalysts are able to migrate into the grain or sugar crop, while at the same time, the desired end-product is preferentially unable to migrate out of the kernel or sugar crop. Therefore the process of fermentation takes place inside the intact kernel or hull of the grain or sugar crop. This process can be called in-situ fermentation. The desired end-products are contained by the intact kernel or hull from diffusion and subsequent evaporative loss, thus improving yield of desirable end-products Further, the desired end-product is not susceptible to further activity by native organisms, thus imparting stability.

This in-situ fermentation therefore preserves the metabolizable carbohydrates in the form of the economically desired end-product, thus alleviating the need for forced-drying. This in-situ fermentation simultaneously reduces the need for further fermentation to obtain economically desirable end-products, providing an important economic benefit.

A desirable feature of the instant disclosure is that, in contrast to preceding methods of imparting stability, higher moisture in the storage environment tends to drive the desirable end products, e.g. ethanol, into the cereal grain or sugar crop. Maintenance of the stabilized product could therefore be optionally further aided by controlling the relative humidity of the surrounding environment, such that moisture tends to accumulate rather than evaporate from the cereal grain kernels or sugar crops.

Also, to ensure that carbohydrate stabilization by a biological catalyst preferentially forms the product of interest, anti-microbial agents can be used to minimize the activity of native microbes.

Thus, it is an object of the present disclosure is to provide, in combination with the other features and advantages disclosed herein, a biologically-stabilized biomass suitable for production of renewable materials that does not require forced drying to impart stability.

A further object of the present disclosure is to provide, in combination with the other features and advantages disclosed herein, a biologically-stabilized biomass that additionally requires less energy and water to convert into a renewable material than conventionally stabilized biomass.

A still further object of the present disclosure is to provide, in combination with the other features and advantages disclosed herein, a method of producing the biologically-stabilized biomass.

Yet another object of the present disclosure is to provide in combination with the other features and advantages disclosed herein, a biomass treatment unit designed to biologically stabilize biomass according to the present disclosure.

### BRIEF DESCRIPTION OF THE DRAWINGS

In the detailed description that follows, reference will be made to the following figures:

FIG. 1 is a schematic view of a preferred embodiment of a processing unit;

FIG. 2 is a schematic view of another preferred embodiment of a processing unit;

FIG. 3 is a data plot of concentrations of fermentation products over time for in situ fermentation; and

FIG. 4 is a data plot of concentrations of soluble sugars over time for in situ fermentation.

### DETAILED DESCRIPTION OF PREFERRED EMBODIMENTS

The disclosure is directed to biologically-stabilized biomass and methods of producing stabilized biomass from biomass suitable for use in the production of renewable material from the stabilized biomass.

In a specific embodiment, the instant disclosure relates to an improvement in processing crops for use as biomass feedstock in ethanol production. Crops harvested for this purpose include grains, sugarcane, Jerusalem artichoke and sugar beets. These biomass feedstocks include, among other compounds, monosaccharides, disaccharides, and oligosaccharides that are desired for eventual conversion by yeast to ethanol.

However, at harvest, native organisms reside on the hull or husk of these crops, and using water that is present at harvest, degrade the monosaccharides, disaccharides, and oligosaccharides into undesirable organic acids. Examples of these native organisms include organisms selected from the genus Lactobacillus and organisms selected from the genus Clostridium.

Consequently, within about 48 hours, these crops are currently processed to dry them, thereby preventing this degradation and protecting the biomass for future conversion to ethanol.

Presently, the drying is performed by at least one of two methods: (a) using high-temperature, low residence-time dryers or (b) using forced-draft fans without adding heat. Each of these methods has drawbacks. Using high-temperature, low residence-time dryers effectively and rapidly drives off water, but the high temperatures encountered therein permit conversion of a portion of the monosaccharides, disaccharides, and oligosaccharides into char or Maillard-type reaction products, neither of which can then be fermented by yeast. Using forced-draft fans without heat to drive off water avoids the unwanted elevation in temperature, but suffers from requiring a relatively long residence time, during which the native organisms degrade a portion of the monosaccharides, disaccharides, and oligosaccharides into undesirable organic acids.

Thereafter, these crops are milled. Water is then added, along with yeast. These crops are thereby fermented for ethanol production as the yeast uses water to metabolize the monosaccharides, disaccharides, and oligosaccharides present in the crops into ethanol.

The instant disclosure provides a method of preventing undesirable degradation of these crops, whereby the yeast to be used to ferment the monosaccharides, disaccharides, and oligosaccharides in the crops is applied to these crops before the crops are milled. In a more specific embodiment, the yeast is applied to the crops within 48 hours of harvest. Thus, the crops do not need to be milled before applying the yeast, and optionally, they do not even need to be dried.

Thus, in the preferred embodiments, the grain kernels or the hulls of the biomass feedstock remain intact when the yeast is applied. The yeast out-competes the native organisms for the monosaccharides, disaccharides, and oligosaccharides, fermenting them into desirable ethanol. The native organisms do not metabolize ethanol. Further, the intact kernels or hulls are semipermeable to ethanol and yeast. The kernels or hulls permit yeast into the grain, sugarcane, Jerusalem artichoke, or sugar beets, but do not allow ethanol to evaporate.

Therefore, applying yeast to the undried, whole grain kernels and whole sugarcane, Jerusalem artichoke and sugar beets, simultaneously (a) prevents degradation of monosaccharides, disaccharides, and oligosaccharides into undesirable organic acids by bacteria, (b) increases the yield of ethanol and (c) eliminates the need to mill these crops and, if done within 48 hours of harvest, (d) eliminates the need to dry the crops.

A convenient way to determine if desirable fermentation by yeast, rather than undesirable degradation by native organisms, has taken place in the biomass feedstock is to determine the average oxidation number of the carbon atoms in the biomass feedstock. The monosaccharides, disaccharides, and oligosaccharides present in the crops at harvest have an oxidation number of 0. The desirable ethanol has an oxidation number of −2. The undesirable organic acids have oxidation numbers greater than 0. Therefore, if sufficient, desirable fermentation has taken place, the average oxidation number of the carbon atoms in the in the biomass feedstock will be less than 0. Conversely, if excessive, undesirable degradation by native organisms has taken place, the average oxidation number of the carbon atoms in the biomass feedstock will be greater than 0. A biomass feedstock that has neither been fermented by yeast, nor degraded by native organisms, will have average oxidation number of the carbon atoms of 0.

Thus, according to this disclosure, the biomass is converted into biologically-stabilized biomass by inoculating the biomass with a biological catalyst, yeast. The inoculation is carried out by treating the biomass with high-density formulations of the biological catalyst by e.g. spraying, wet-soak, drum mixers, table mixers, etc.

According to some embodiments, the disclosure is directed towards methods of producing biologically-stabilized biomass suitable for use in the production of renewable materials. The method includes consuming at least a portion of a biomass with an inoculating biological catalyst to produce at least a first renewable material. The method includes consuming and producing substantially inside a kernel or outer hull of the biomass comprising active matter, inactive matter, and water. Importantly, the biomass is not force-dried to effect stabilization.

Biomass

Biomass used as fermentation feedstock comprises plant and/or animal material derived at least in part from living organisms or from recently living organisms which is comprised of water, active matter and inactive matter.

According to different embodiments, the biomass comprises (a) grain; (b) sugarcane; (c) sugar beets; (d) Jerusalem artichoke and/or (e) lignocellulose.

According to different embodiments, the biomass has not been substantially force-dried.

According to different embodiments, the biomass has moisture content of (a) between about 18% and about 50%; (b) between about 20% and about 30%; or (c) between about 22% and about 25%.

Active Matter

Active matter comprises compounds that may be further converted into an intended desirable renewable end product by a biological catalyst, as well as products (desirable and undesirable) of metabolism by biological catalysts and/or undesirable native microbes.

In different embodiments, the active matter includes: (a) mono-, di-, and/or oligo-saccharides; (b) hydrogen; (c) hydrocarbons; (d) alcohols; (e) organic acids; (f) diols; (g) isoprenoids, (h) polyhydroxyalkanoates.

Oxidation State of Carbon Atoms in Active Matter

The average oxidation state or oxidation number of carbon in active matter is an important determinant of overall stability and potential energy yield of the active matter in the biomass.

The oxidation state of carbon in a given species indicates the relative oxidative distance between the fully reduced carbon atom and the fully oxidized carbon atom. For example, methane [CH₄] has an oxidation number of −4, and carbon dioxide [CO₂] has an oxidation number of +4.

The average oxidation state of carbon atoms in various organic molecules is found by using the elemental composition of the compound or mixture, combined with certain rules, which account for the charges associated with the non-carbon atoms in a given molecule.

Table 1 shows the oxidation state of various atoms that can be used for initially assigning oxidation state to the carbon atoms in certain molecules.

TABLE 1
Oxidation state of various atoms

| | |
|---|---|
| Hydrogen | +1 |
| Oxygen | −2 |
| Sulfur | −2 |
| Nitrogen | −3 |

Highly metabolisable mono-, di- and oligo-saccharides (also called sugars) photosynthesized from carbon dioxide represent solar energy which has transformed carbon from an oxidation state of +4 (CO2) to zero (sugars). The process of metabolism, like that of fuel combustion, produces energy by the re-oxidation of relatively reduced substances.

These highly metabolisable saccharides are susceptible to undesirable decay or degradation by native microbes. In general, decay or degradation is the formation of products, such as organic acids by contaminating and by native bacteria. These undesirable degradation products have higher oxidation numbers of carbon, which means that they provide relatively less energy when re-oxidized to carbon dioxide. Therefore, these undesirable degradation products having higher oxidation numbers of carbon are not considered to be desirable renewable end-products.

Mono-, di-, and oligo-saccharides have average carbon oxidation numbers of about zero. Undesirable products of degradation, such as organic acids, acetate, citrate or malate have average carbon oxidation state greater than or equal to zero. Desirable renewable end-products, such as alcohols, including ethanol, butanol, and propanol, have average carbon oxidation state less than zero.

Conversion of saccharides from a carbon oxidation state of zero to desirable, renewable end-products, which have carbon oxidation numbers less than zero, represents the preservation of the original solar energy in a chemical species that provides relatively more energy when re-oxidized to carbon dioxide, as when burning it for fuel.

The average oxidation number of carbon in various organic species found in the active matter of biomass, based on the rules shown in Table 1, combined with elemental analysis, is shown in Table 2.

TABLE 2
Average Oxidation State of Carbon Atoms
in the Active Matter of Biomass

| | Average Oxida- | |
|---|---|---|
| | tion State of | |
| Molecule | Carbon Atoms | Type of Molecule |
| glucose | 0.00 | Saccharide |
| fructose | 0.00 | Saccharide |
| galactose | 0.00 | Saccharide |
| mannose | 0.00 | Saccharide |
| xylose | 0.00 | Saccharide |
| xylulose | 0.00 | Saccharide |
| psicose | 0.00 | Saccharide |
| sorbose | 0.00 | Saccharide |
| tagatose | 0.00 | Saccharide |
| lyxose | 0.00 | Saccharide |
| arabinose | 0.00 | Saccharide |
| erythrose | 0.00 | Saccharide |
| erythrulose | 0.00 | Saccharide |
| ribose | 0.00 | Saccharide |
| ribulose | 0.00 | Saccharide |
| threose | 0.00 | Saccharide |
| allose | 0.00 | Saccharide |
| gulose | 0.00 | Saceharide |
| idose | 0.00 | Saccharide |
| lactose | 0.00 | Saccharide |
| sucrose | 0.00 | Saccharide |
| cellobiose | 0.00 | Saccharide |
| xylobiose | 0.00 | Saccharide |
| maltose | 0.00 | Saccharide |
| trehalose | 0.00 | Saccharide |
| raffinose | 0.00 | Saccharide |
| stachyose | 0.00 | Saccharide |
| verbascose | 0.00 | Saccharide |
| galactinol | 0.00 | Saccharide |
| hydrogen | N/A | Desirable Renewable End-Product |
| methane | −4.00 | Desirable Renewable End-Product |
| ethane | −3.00 | Desirable Renewable End-Product |
| ethylene | −2.00 | Desirable Renewable End-Product |
| butylene | −2.00 | Desirable Renewable End-Product |
| methanol | −2.00 | Desirable Renewable End-Product |
| ethanol | −2.00 | Desirable Renewable End-Product |
| propanol | −2.00 | Desirable Renewable End-Product |
| butanol | −2.00 | Desirable Renewable End-Product |
| formaldehyde | 0.00 | Desirable Renewable End-Product |
| acetaldehyde | −1.00 | Desirable Renewable End-Product |
| butyraldehyde | −1.50 | Desirable Renewable End-Product |
| propionaldehyde | −1.33 | Desirable Renewable End-Product |
| propanediol | −1.33 | Desirable Renewable End-Product |
| butanediol | −1.50 | Desirable Renewable End-Product |
| polyhydroxy- | −0.50 | Desirable Renewable End-Product |
| alkanoate (PHB) | | |
| isoprene | −1.60 | Desirable Renewable End-Product |
| acetic acid | 0.00 | Undesirable Degradation Product |
| propionic acid | −0.67 | Undesirable Degradation Product |
| lactic acid | 0.00 | Undesirable Degradation Product |
| butyric acid | −1.00 | Undesirable Degradation Product |
| citric acid | 1.00 | Undesirable Degradation Product |
| malic acid | 1.00 | Undesirable Degradation Product |
| maleic acid | 1.00 | Undesirable Degradation Product |
| malonic acid | 1.33 | Undesirable Degradation Product |
| succinic acid | 0.50 | Undesirable Degradation Product |
| fumaric acid | 1.00 | Undesirable Degradation Product |
| oxaloacetic acid | 1.50 | Undesirable Degradation Product |
| ketoglutaric acid | 0.80 | Undesirable Degradation Product |
| isocitrate | 1.00 | Undesirable Degradation Product |
| aconitic acid | 1.00 | Undesirable Degradation Product |
| formic acid | 2.00 | Undesirable Degradation Product |

The average oxidation state of the carbon atoms in the active matter corresponds to the degree of oxidative degradation of the carbon-containing molecules in the active matter.

Average carbon oxidation state of the carbon atoms in the active matter of the biomass therefore is an indicator of the viability of active matter for the further production of renewable materials. The carbon oxidation state also is an indicator of active matter stability prior to the further production of renewable materials.

Further, the average oxidation state of the carbon atoms in the active matter of the biomass distinguishes between undesirable degradation by native species and desirable fermentation by the inoculating biocatalysts. Undesirable degradation by native species tends to produce acidic moieties, in which the average oxidation state of the carbon atoms is greater than 0, while desirable fermentation by the inoculating biocatalysts tends to produce species, e.g. alcohols, in which the carbon atoms have an average oxidation state less than 0.

The average oxidation number (AO#) of carbon in active matter is found by taking the number-weighted average of the oxidation number of each species in active matter. More specifically, the average oxidation number of carbon in active matter is found by:

AO#=Σ[Wi÷MWi*nci*ONCi]÷Σ[Wi÷MWi*nci] (1)

Where:
Wi=wt % in biomass of species i
MWi=Molecular Weight of species i
nci=number of carbon atoms in species i
ONCi=oxidation number of carbon in species i, as shown in Table 2

In different embodiments, the active matter includes carbon atoms with an average oxidation state of less than about (a) 0.0; (b) negative 0.25; (c) negative 0.50; (d) negative 0.75; (e) negative 1.00; (f) negative 1.25; (g) negative 1.50; (h) negative 1.75; (i) negative 2.00; (k) negative 2.25; (l) negative 2.50; (m) negative 2.75; (n) negative 3.00; (o) negative 3.25; (p) negative 3.50; (q) negative 3.75; or (r) negative 4.00.

Inactive Matter

Inactive matter comprises materials in the biomass which will not be converted into economically desirable renewable end products.

In different embodiments, the inactive matter may include (a) polysaccharides; (b) lipids; (c) amino acids; (d) genetic material; (e) phenolic lignin compounds; and/or (f) ash, e.g. sodium, magnesium, silicon, phosphorous, potassium or zinc.

Conversion of Inactive Matter to Active Matter

According to one embodiment the method includes converting a portion of the inactive matter to active matter in order to increase the yield of desirable renewable materials. Examples of conversion may include enzyme hydrolysis, acid-catalyzed hydrolysis, base-catalyzed hydrolysis, and the like.

According to another embodiment the inactive matter is further processed to produce a renewable material. Examples of further processing may include storing, milling, fractionating, steeping, slurrying, liquefying, digesting, fermenting, degassing, distilling, purifying, drying, or combinations thereof.

Currently, the feedstock is subjected to these processes prior to inoculation with a biological catalyst intended to convert active matter into desirable renewable materials. Therefore, there is still opportunity for the native organisms to degrade this newly created active matter. However, according the method of the instant disclosure, an inoculating biocatalyst is already present in the feedstock and so desirable fermentation can occur immediately, thus preventing native organisms from degrading the newly-created active matter. This improves the yield of desirable renewable materials.

Inoculating Biological Catalyst

The inoculating biological catalyst is a suitable fermentation organism which produces the intended economically desirable renewable end product. According to different embodiments, the inoculating biological catalyst comprises (a) a yeast; (b) a cyanobacteria, fungus or algae; (c) diatom or phytoplankton; (d) a genetically modified organism; (e) an ethanologenic yeast such as Saccharomyces cerevisiae, Saccharomyces pastorianus, Saccharomyces carlsbergensis, Saccharomyces bayanus, Saccharomyces paradoxus.

Biologically-Stabilized Biomass

Biologically-stabilized biomass according to the present disclosure comprises biomass that has been treated with an inoculating biological catalyst which produces an intended renewable end-product by fermentation. Such fermentation preserves mono-, di-, and oligo-saccharides in the form of the economically desired end-product, thus alleviating the need for extensive forced-drying. This fermentation simultaneously reduces the need for further fermentation to obtain economically desirable end-products. Additionally, because the biomass has not been extensively dried, there is a reduced need to add water when effecting the further fermentation to obtain economically desirable end-products. Finally, the desired end-products are not susceptible to further activity by native organisms, thus imparting stability even in the presence of these native organisms.

According to some embodiments the native organism comprises (a) a bacteria; (b) organisms selected from the genus Lactobacillus; and/or (c) organisms selected from the genus Clostridium.

According to different embodiments, the biologically-stabilized biomass includes fewer than X colony forming units (CFU) of native organism per gram, where X is equal to (a) 10,000; (b) 5,000; (c) 1,000; (d) 500; (e) 250 or (f) 100.

According to some embodiments, the biologically-stabilized biomass includes at least Y CFU of inoculating biological catalyst per gram, where Y is equal to (a) 100; (b) 250; (c) 500; (d) 1,000; (e) 5,000; or (f) 10,000.

Anti-Microbial Agents

According to some embodiments, the biologically-stabilized biomass may contain anti-microbial agents, designed to preferentially minimize the activity of native microbes, while permitting the inoculating biological catalysts to impart stabilization to the biomass. Such anti-microbial agents can include antibiotics (chloramphenicol, ampicillin, penicillin, tetracycline, erythromycin, amoxicillin), disinfectants (bleach, hydrogen peroxide, ozone, sodium bicarbonate), metals (copper, zinc, silver), oils (cinnamon oil, clove oil, eucalyptus oil, onion oil, lemon oil, lavender oil, mint oil), sulfonamides (sulfamethoxazole, sulfisomidine, sulfadiazine, sulfadoxine, sulfacetamide, dichlorphenamide, sulfonylurea, thiazide, chlorothiazide, hydrochlorothiazide).

Antimicrobial activity can also be achieved by using heat or radiation.

According to some embodiments, the anti-microbial agents are present at 1-999 parts per billion. According to some other embodiments, the anti-microbial agents are present at 1-999 parts per million.

Desirable Renewable End Products

According to some embodiments, renewable materials may include hydrogen, methane, ethane, ethylene, butylene, methanol, ethanol, propanol, butanol, formaldehyde, acetaldehyde, butyraldehyde, propionaldehyde, propanediol, butanediol, polyhydroxyalkanoates, isoprenoids, or any combination thereof.

Methods of Producing Stabilized Biomass

Stabilized biomass is produced by consuming at least a portion of a fermentation feedstock with a biological catalyst to produce a renewable material, wherein the consuming occurs substantially inside the whole kernel or outer hull of the fermentation feedstock.

In some embodiments, fermentation feedstocks include whole-kernel corn with moisture content greater than 16 weight %.

In accordance with certain embodiments of the disclosure, the amount of inoculating biological catalyst added to the fermentation feedstock is a percentage of the dry weight of the fermentation feedstock. The inoculating biological catalyst can also be added to the fermentation feedstock as a percentage of the undried corn.

In one embodiment, Saccharomyces cerevisiae is added at 0.01% dry basis (of the weight of dry whole-kernel corn).

In other embodiments, Saccharomyces cerevisiae is in solution at a concentration of between about 5 and about 300 grams per Liter.

According to one embodiment, a 1.0 weight % biocatalyst inoculation is achieved by mixing 200 milliliters of 50 grams per Liter Saccharomyces cerevisiae solution with 1 kilogram of 20 weight % moisture whole-kernel corn.

In one embodiment, the fermentation feedstock is inoculated with biological catalyst by mixing in a rotary drum mixer or in a paddle mixer.

In another embodiment, the fermentation feedstock is inoculated with biological catalyst by spraying the biological catalyst onto the fermentation feedstock, such as on a moving bed, on a table mixer, or by spraying biological catalyst on to a falling stream of the fermentation feedstock.

In another embodiment, the fermentation feedstock is inoculated with biological catalyst by mixing 200 milliliters of 50 grams per Liter Saccharomyces cerevisiae solution with 1 kilogram of dry whole-kernel corn in a 1-gallon plastic flexible container and tumbling end-over-end by hand until thoroughly mixed, such as for one minute. Optionally, any excess inoculation solution is separated from the inoculated feedstock, such as by filtration, decanting, gravity separation or the like.

In accordance with certain embodiments of the disclosure, inoculated fermentation feedstock is stored for a residence time, wherein the biological catalyst consumes at least a portion of the fermentation feedstock to produce a renewable material, and wherein the consuming occurs substantially inside the fermentation feedstock.

According to some embodiments the residence time is sufficient to convert at least a portion of the active matter in the fermentation feedstock to a renewable material.

According to some embodiments, the produced renewable material includes an alcohol.

According to one embodiment, the carbon atoms in the renewable material have average oxidation state of negative two (−2). According to another embodiment, the average oxidation state of the carbon atoms in the biologically stabilized biomass is less than 0.

Biomass Treatment Unit

According to some embodiments, the disclosure is directed towards a biomass treatment unit. The biomass treatment unit is intended to impart stabilization to the biomass utilizing the instant disclosure, as well as to effect inoculation of the biomass, therefore reducing the need for additional biological catalyst added at later stages in processing the feedstock into desirable renewable materials.

The biomass treatment unit includes a series of stages from a first stage to a final stage, with one or more intermediate stages there between, wherein the first stage comprises an inlet for unstabilized biomass. The biomass treatment unit includes one or more systems that continually apply one or more treatments to one or more intermediate stages and a system that continually moves the biomass from the first stage to the final stage, thereby producing a biologically-stabilized biomass stream.

According to some embodiments the intermediate-stages include rinsing, disinfecting, drying, humidifying, inoculating with inoculating biological catalysts according to the instant disclosure or combinations thereof.

According to different embodiments, the number of intermediate stages is 1, 2, 3, 4, 5 or 6.

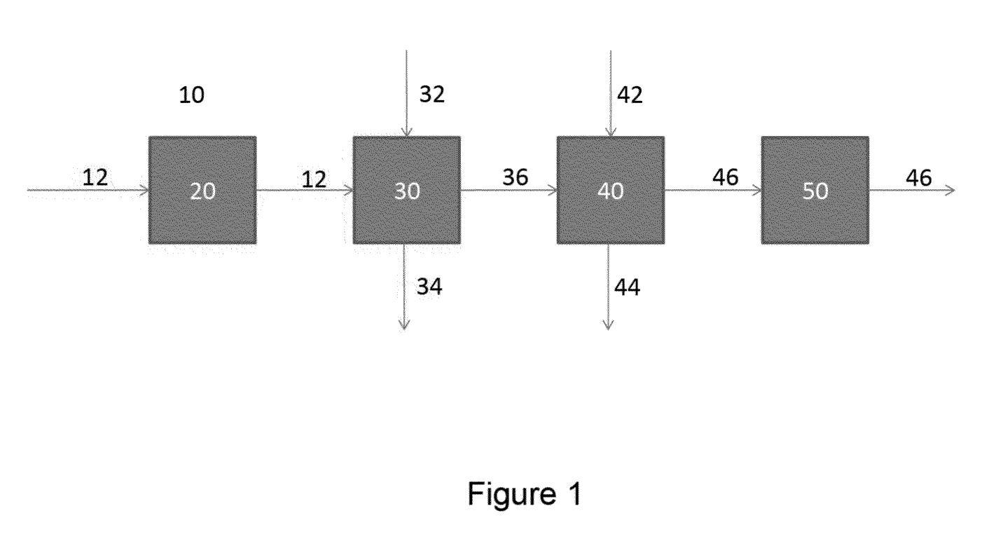

As shown in FIG. 1, an exemplary treatment unit is illustrated. Untreated biomass 12 enters Stage 1 (20) and is conveyed to Stage 2 (30), which comprises a washing stage. In the washing stage 30, a washing solution 32 enters and is mixed with the untreated biomass 12. The washing solution 32 may contain water and optionally contains an antimicrobial agent. Spent washing solution 34 exits the washing stage 30. A washed biomass stream 36 is conveyed from the washing stage 30 to an inoculation stage 40. In the inoculation stage 40, inoculation solution 42 is mixed with the washed biomass stream 36. A spent inoculation stream 44 exits the inoculation stage 40. A washed, inoculated biomass stream 46 exits the inoculation stage 40 and is sent to a fourth stage 50.

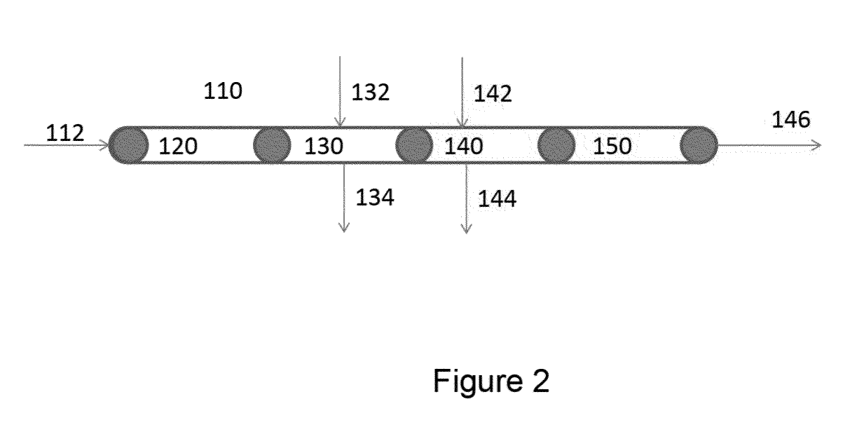

Referring now to FIG. 2, a more preferred embodiment comprises a moving bed, vacuum-filtration system 110. Untreated biomass 112 enters Stage 1 (120) and is conveyed to Stage 2 (130), which comprises a washing stage. In the washing stage 130, a washing solution 132 enters and is mixed with the untreated biomass 112. The washing solution 132 may contain water and optionally contains an antimicrobial agent. Spent washing solution 134 exits the washing stage 130. A washed biomass stream 136 is conveyed from the washing stage 130 to Stage 3, an inoculation stage 140. In the inoculation stage 140, inoculation solution 142 is mixed with the washed biomass stream 136. A spent inoculation stream 144 exits the inoculation stage 140. A washed, inoculated biomass stream 146 exits the inoculation stage 140 and is sent to a fourth stage 150.

### Example

### Biological Stabilization and In-Situ Fermentation with Saccharomyces Cerevisiae of Moist, Whole Kernel Zea mays Contaminated with Native Microbes

Summary:

A series of experiments were conducted to show that the ethanologenic yeast Saccharomyces cerevisiae is capable of biological stabilization of the soluble sugars in mature whole kernels of Zea mays that are moist and contaminated with native microbes, thus showing that Saccharomyces cerevisiae is an effective biological stabilizer for a fermentable biomass. Importantly, at higher moisture contents, and therefore more unstable, whole-kernel corn resulted in higher ethanol concentrations than similarly high-moisture broken kernels, showing that Saccharomyces cerevisiae is especially suitable as a biological stabilizer for undried, whole kernel corn contaminated with native microbes. A time-course fermentation experiment on whole kernel, contaminated corn showed that the ethanol concentration reached 0.16 weight percent without appreciable acid formation, and that fermentation of residual sugars was continuing 176 hours after inoculation.

First, initial validation experiments were conducted on freshly-harvested corn kernels having 20%, 22% or 24% moisture to evaluate the impact of moisture content and to evaluate the impact of contamination and whole vs. broken kernels on sugar conversion at these three moisture contents.

The effect of drying was also investigated because the 20% moisture sample was obtained by drying a sample of 22% moisture corn using a forced-draft fan for about four hours. The 22% moisture and 24% moisture samples of corn were not dried.

To evaluate the effect of washing, the whole kernels were either washed to remove contaminant native microbes, or were left unwashed, so that the contaminant native microbes were present in the corn.

Samples of each of the unwashed and washed kernels were ruptured in order to compare them with unwashed and washed whole kernels. These samples were fermented and the levels of sugars, acids and ethanol compared to determine the efficacy of Saccharomyces cerevisiae as a biological stabilizer and fermenting catalyst on unwashed vs. washed corn, on whole kernels vs. ruptured kernels and on dried vs. undried corn.

Finally, a time-course of in-situ fermentation was conducted on corn at 22% moisture. Samples of unbroken and unwashed kernels with 22% moisture content were inoculated with Saccharomyces cerevisiae in triplicate for each time-point. Triplicate samples were allowed to ferment and were subsequently analyzed for composition after 26, 50, 70 and 176 hours.

Materials and Methods

Feedstock and Stabilization Organisms:

The Zea mays feedstock for this study was Fontanelle Hybrid 4A503RBC with Monsanto Genuity Smartstax traits. Corn of 97-day relative maturity was harvested between Sep. 27 and Oct. 14, 2013 from Johnson County, Iowa. Corn was harvested by hand-picking ears (with gloves) and a mechanical sheller was used to remove the kernels from the cob. To achieve 20% moisture, a sample of 22% moisture corn was dried using a forced-draft fan for about four hours. The moisture content of the harvested corn was verified using a Dickey John GAC II grain-moisture tester. Harvested corn samples were held at −10° C. before thawing at 4° C. overnight for use.

The biological stabilizer used for this study was Fleischmann-brand active-dried baker's yeast. Unless specified otherwise, one individually-wrapped, 7 g package of active-dried yeast was used for each inoculation.

Feedstock Treatments:

Approximately 300 mL of moist corn was used for each treatment, equivalent to 0.5 kg at an average bulk density of 25 kg per bushel. The general order of treatments was as follows: washing, breaking, inoculation, fermentation. Where indicated, corn samples were washed by adding 300 mL of sterile water to the corn in a 1 quart bag, and mixing end-over-end for 1 minute. Wash water was then decanted completely and the corn used for further treatment. Where indicated, corn samples were broken by placing the 1 quart bag containing corn between two metal plates, and applying downward pressure until the majority of kernels showed visible deformation. Corn samples were inoculated by combining one 7 g package of active-dried yeast to 300 mL of sterile water. The yeast solution was then added to the 1 quart bag, and the corn was inoculated by mixing end-over-end for about 1 minute. The yeast solution was then decanted completely, and the inoculated corn was used in batch fermentations. Batch fermentations were conducted by adding inoculated corn to individual, sterile 10 ounce Nuby-brand baby bottles with a total capacity of approximately 300 mL. No attempt was made to replace the residual pockets of air between corn kernels with an anaerobic atmosphere. The bottles were capped and stored at room temperature for the desired residence time, and then stored at 4° C. until HPLC analysis of carbohydrate concentrations.

Analytical Methods:

Analytical analysis was conducted by Global Pharma Analytics (Jupiter, Fla.). HPLC (Perkin-Elmer LC 200) was used to evaluate the products of fermentation, and to evaluate the impact of whole vs. broken kernels and contaminated vs. uncontaminated corn on the biological stabilization of soluble sugars. Treated and control samples of corn were prepared for analysis by mixing 40 grams of corn (approximately 15-20% of the total sample) with 40 grams of reverse osmosis purified water and blending to a slurry for about five minutes in a Hamilton Beach blender. Supernatant liquid was then decanted and filtered through a 45 micron filter-disk. Filtered supernatants were analyzed by HPLC using a Biorad HPX-87H Aminex column, with a mobile phase of 0.005M H2SO4 at 1.0 mL/minute. The resulting peak areas were used to estimate carbohydrate concentrations including sucrose, glucose, fructose, acetic acid, lactic acid and ethanol by comparison with peak areas for standards of known concentration.

Results:

Table 3 shows the concentrations of the active matter (in this case, ethanol, acetate, lactate, glucose, fructose and sucrose) measured in corn samples 1, 2 and 3 which were submitted directly for analysis without any treatment (unbroken and unwashed). Table 3 also shows the average oxidation state of carbon atoms in the active matter of these samples, calculated using Table 2 and Equation (1).

TABLE 3
Profile of carbohydrates measured in unbroken, unwashed control corn samples.
Values expressed as a percentage of the total kernel wet-weight.

| | | | | | | | | Average |
|---|---|---|---|---|---|---|---|---|
| | Moisture | Ethanol | Acetate | Lactate | Glucose | Fructose | Sucrose | oxidation |
| Sample | (%) | (%) | (%) | (%) | (%) | (%) | (%) | state |
| 1 | 20 | 0.001 | 0.000 | 0.000 | 0.209 | 0.244 | 0.077 | −0.005 |
| (dried) | | | | | | | | |
| 2 | 22 | 0.003 | 0.000 | 0.001 | 0.134 | 0.141 | 0.252 | −0.014 |
| 3 | 24 | 0.001 | 0.002 | 0.067 | 0.000 | 0.011 | 0.200 | −0.009 |

Samples of all three moisture contents contained detectable amounts of sucrose. The 20% and 22% moisture corn samples also contained detectable levels of glucose and fructose. The sample of 24% moisture corn did not contain measurable amounts of glucose and only a trace amount of fructose, while at the same time there was a greater amount of lactate detected than in the 20% and 22% moisture corn samples. This suggests that the 24% moisture corn contained a greater amount of active, contaminant organisms, which may have partially fermented the available soluble sugars.

The average oxidation state of the carbon atoms in the active matter of all three samples was very slightly negative.

Impact of Moisture Content and Pretreatment on In-Situ Fermentation of Soluble Sugars:

Table 4 shows the carbohydrate concentrations from an initial, single-replicate experiment conducted to explore the impact of kernel moisture content and whole vs. broken kernels and contaminated vs. uncontaminated feedstock on the fermentability of soluble sugars inside the kernel.

TABLE 4
Impact of pretreatments on the profile of carbohydrates detected after 51 hours of in-situ fermentation
at 24° C. of soluble sugars. Values shown are expressed as a percentage of the total kernel wet-weight.

| | | | | | | | | | | Average |
|---|---|---|---|---|---|---|---|---|---|---|
| | Moisture | Washed | Whole | Ethanol | Acetate | Lactate | Glucose | Fructose | Sucrose | oxidation |
| Sample | (%) | (Y or N) | (Y or N) | (%) | (%) | (%) | (%) | (%) | (%) | state |
| 4 | 20 | N | Y | 0.113 | 0.003 | 0.000 | 0.0292 | 0.469 | 0.051 | −0.420 |
| (dried) | | | | | | | | | | |
| 5 | 20 | N | N | 0.133 | 0.008 | 0.007 | 0.117 | 0.232 | 0.032 | −0.607 |
| (dried) | | | | | | | | | | |
| 6 | 20 | Y | Y | 0.366 | 0.006 | 0.008 | 0.004 | 0.060 | 0.002 | −1.712 |
| (dried) | | | | | | | | | | |
| 7 | 22 | N | Y | 0.422 | 0.024 | 0.003 | 0.000 | 0.015 | 0.000 | −1.857 |
| 8 | 22 | N | N | 0.386 | 0.018 | 0.007 | 0.000 | 0.019 | 0.000 | −1.838 |
| 9 | 22 | Y | Y | 0.367 | 0.017 | 0.028 | 0.010 | 0.000 | 0.000 | −1.792 |
| 10 | 24 | N | Y | 0.468 | 0.024 | 0.044 | 0.021 | 0.016 | 0.000 | −1.704 |
| 11 | 24 | N | N | 0.121 | 0.026 | 0.004 | 0.023 | 0.000 | 0.000 | −1.494 |
| 12 | 24 | Y | Y | 0.190 | 0.015 | 0.003 | 0.010 | 0.000 | 0.000 | −1.795 |

At each available moisture content, one set of samples was unwashed and whole prior to inoculation (samples 4, 7 and 11), one set of samples was unwashed but was broken (samples 5, 8 and 12), while yet another set was washed but unbroken (samples 6, 9 and 12) prior to inoculation. Inoculated samples were held at 24° C. to ferment for 51 hours before storage at 4° C. to halt fermentation until HPLC analysis was performed.

Examining the data presented in Table 4, the effect of whole vs. broken kernels and unwashed vs. washed whole kernels on final ethanol concentrations and average oxidation state of carbon atoms in the active matter is apparent, especially at higher moisture content. At 22% and especially at 24% moisture, the ethanol concentration was higher and the average oxidation state tended to be more negative for unbroken kernels than for broken ones.

At 20% moisture (dried samples), the final ethanol concentrations were comparable between unbroken and broken kernels that had not been washed (Samples 4 and 5). Significantly, Sample 6 (dried, washed, unbroken) had the highest ethanol concentration of the three 20% moisture samples, as well a significantly more negative average oxidation number than the other two samples, indicating that the additional moisture from the washing step as well as the whole kernels combined to improve the yield of desirable species in a previously dried sample, as indicated by the average oxidation state and the ethanol level. This is contrary to current practice, where additional moisture is considered to contribute to undesirable decomposition.

Additionally, the higher moisture content (and thus normally more susceptible to degradation by native microbes) whole kernels that were unwashed had higher final ethanol concentrations than the washed samples, showing that the S. cerevisiae is an effective biological stabilizer, because in the presence of moisture it is able to out-compete the native microbes to metabolize the mono- and di-saccharides, especially in whole-kernel corn.

Improved ethanol production with whole kernel corn is likely due to differences in mass transfer between whole and broken kernels. The broken kernels simultaneously allow improved transfer of oxygen into the kernels and oxygen out of the kernels. The higher oxygen content contributes to increased degradation at the expense of fermentation, while the ethanol generated can more readily evaporate from the broken kernels than from the whole kernels.

Breaking the kernels open prior to inoculation with S. cerevisiae did not appear necessary for fermentation of soluble sugars (sucrose, glucose, fructose). Broken and unbroken samples contained approximately the same residual sugar concentrations at all initial moisture contents tested, but the broken kernels had a lower ethanol concentration.

Looking more closely at the data in Table 4, it is clear that increasing initial kernel moisture content generally resulted in higher overall metabolic activity. Samples containing 20% moisture generally contained lower final concentrations of fermentation products after 51 hours of fermentation than the corresponding samples having 22% or 24% moisture initially. For instance, in Table 4 samples 7 and 11, corresponding to initial moisture contents of 22% and 24%, respectively, showed approximately four times the final ethanol concentration as compared with sample 4 which contained only 20% moisture initially. The amount of organic acids generated also increased as the initial moisture content increased, as samples 7-9 and samples 10-12 showed consistently higher levels of acetate and lactate as compared with samples 4-6. Finally, the concentration of residual sugars was generally highest in the 20% moisture samples, again indicative of decreased metabolic activity when the initial moisture content was as low as 20% and the sample had been dried.

Therefore, it is clear that if corn can be biologically stabilized without lowering the moisture content, according to the instant disclosure, improved fermentation yield of desirable end products can be achieved, as indicated by more negative average oxidation state of carbon atoms in the active matter.

In-Situ Fermentation of Contaminated Whole Kernel Corn:

A final round of experiments to determine the time-course of fermentation was conducted on the whole kernel, unwashed, undried, intermediate moisture level (22%) corn sample. The objective is to show that the biologically stabilized corn can be used as a feedstock to produce ethanol, a desirable renewable material.

Samples of 22% moisture corn were inoculated in triplicate for each desired time point, and held at 18° C. for 26 and 50 hours and then held at 24° C. for 70 and 176 hours.

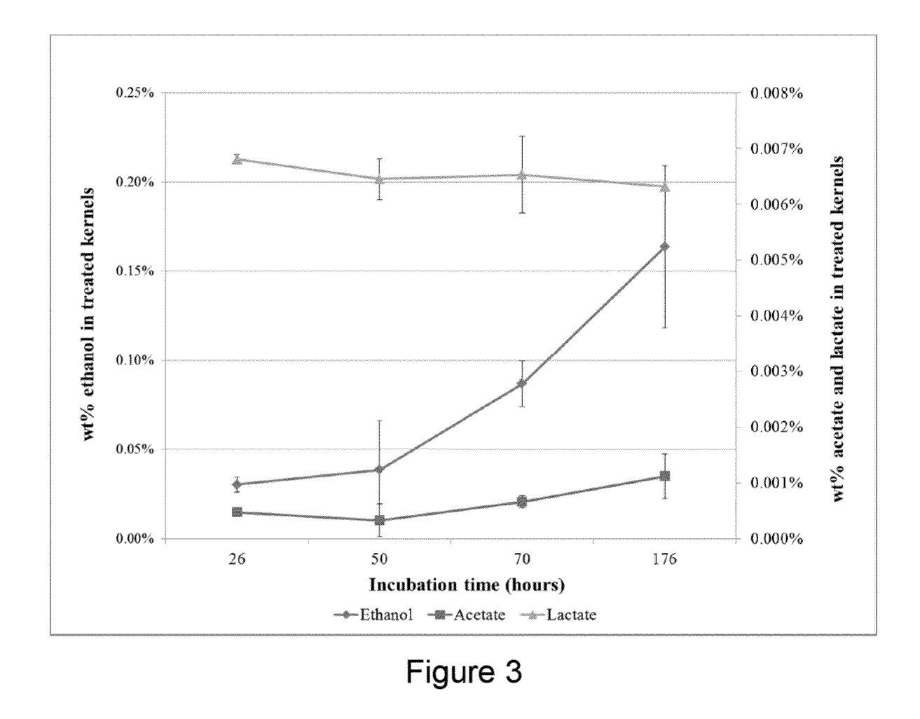

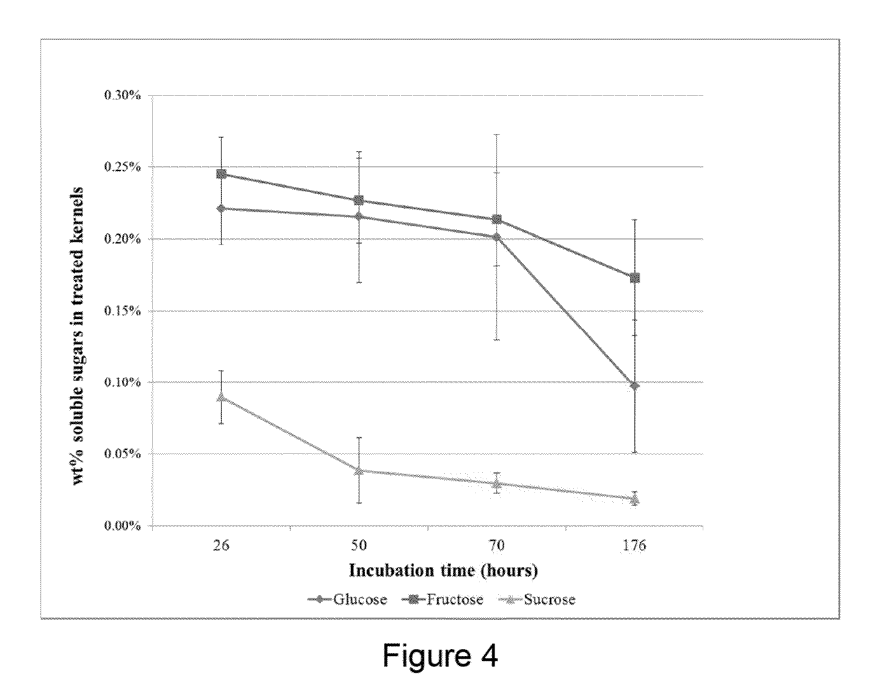

FIGS. 3 and 4 show the resulting carbohydrate concentrations detected during the 176 hour fermentation time course. FIG. 3 shows the concentrations of ethanol and organic acids, while FIG. 4 shows the residual concentrations of soluble sugars detected at each time point. Each data point in FIGS. 3 and 4 represent the average of triplicate inoculations.

The time-course fermentations initiated slowly, with the average ethanol concentration increasing only to 0.04 weight % after 50 hours, compared with up to 0.5 weight % after 51 hours in the initial experiments. This could be due to the slightly reduced room temperature during the first 48 hours of the time course experiment. The fermentation reached exponential phase after 50 hours, and ethanol concentrations increased to an average of 0.164 weight % after 176 hours.

Concentrations of the organic acids lactate and acetate are also shown in FIG. 3. Lactate concentration remained constant at 0.006-0.007 weight % throughout the time course. Acetate concentration was consistently less than 0.001 weight % up to hour 50, but increased linearly after 70 hours and reached a final concentration of 0.005 weight % after 176 hours. These data indicate that native microbial contaminants did not significantly degrade the available sugars over the time course.

Concentrations of soluble sugars including glucose, fructose and sucrose are shown in FIG. 4. Sucrose concentration fell rapidly from 26 to 50 hours, and then declined more gradually over the remaining 126 hours. Conversely, glucose and fructose concentrations decreased only slightly up to hour 70, and then began to decrease more rapidly from hour 70 until the end of the time course. Also, after 70 hours the concentration of glucose fell more rapidly than the concentration of fructose. These data indicate that sucrose was being actively hydrolyzed within the corn kernels to glucose and fructose up to hour 70, likely due to the activity of extracellular invertase enzymes secreted by S. cerevisiae. The data also confirm the preference of yeast for metabolizing glucose over fructose when both are available at equivalent concentrations.

The results of this example show that Saccharomyces cerevisiae is capable of inoculating intact kernels of Zea mays, and can out-compete native microbes for soluble sugars and therefore is an effective biological stabilizer, as indicated by the average oxidation state of carbon in the active matter, especially on wet, undried, unwashed whole kernel corn. Under the conditions tested, S. cerevisiae was capable of alcoholic fermentation of soluble sugars within whole corn kernels, despite the presence of an aerobic atmosphere between kernels.

Overall, these data indicate that in-situ fermentation of soluble sugars by yeast preserved otherwise-labile sugars as the desired end-product of ethanol, thus showing the efficacy of Saccharomyces cerevisiae as a biological stabilizer for wet, whole kernel corn that is contaminated with native microbes. Higher moisture whole kernel corn had higher levels of ethanol and more negative average oxidation state of carbon atoms in the active matter after biological stabilization with Saccharomyces cerevisiae.

Thus, the preceding disclosure provides a stabilized biomass and a method of producing a stabilized biomass. As disclosed, a biomass has active matter containing carbon atoms having an average oxidation state, inactive matter, a biological catalyst having a fermentation organism capable of converting the active matter into a renewable material, and water. The biomass has not been milled. The biomass is suitable for use in the production of renewable materials, such as, for example, ethanol.

More particularly, the biomass can have an average oxidation state of the carbon atoms in the active matter of between −2 and −0.25. The biomass may also contain an antimicrobial agent.

The biological catalyst can be selected from the group consisting of cyanobacteria, fungus, algae, yeast, ethanologenic yeast, diatom and phytoplankton. More particularly, the biological catalyst is ethanologenic yeast, and optionally has between 100 and 10,000 colony forming units of the biological catalyst per gram of biomass.

The biomass can have a moisture content of between 18% and 50% by weight, or preferably a moisture content of between 20% and 30% by weight, or most preferably a moisture content of between 22% and 25% by weight.

The biomass can be a grain, or preferably a whole grain. The grain can be selected from the group consisting of corn, rice, wheat, barley, rye and oats and more particularly from the group consisting of whole corn, whole rice, whole wheat, whole barley, whole rye and whole oats.

Alternatively, the biomass can be selected from the group consisting of sugar beets, sugar cane, Jerusalem artichoke and lignocellulose, or preferably from the group consisting of whole sugar beets, whole sugar cane and whole Jerusalem artichoke.

In one preferred embodiment, the biomass is corn. More preferably, the biomass is corn and the biological catalyst is ethanologenic yeast. Most preferably, the biomass is corn, the biological catalyst is ethanologenic yeast present in the range of between 100 and 10,000 colony forming units per gram of biomass.

In one embodiment, a feedstock for yeast fermentation is disclosed. The feedstock has unmilled, whole kernel Zea mays corn having an initial water content of between 22% and 25% and an initial ethanol content E. The unmilled, whole kernel Zea mays corn has been inoculated with X colony forming units of Saccaromyces cerevisiae yeast per gram of unmilled, whole kernel Zea mays corn within 48 hours of harvest. The whole kernel Zea mays corn further has been fermented by the at least X colony forming units of Saccaromyces cerevisiae yeast per gram of whole kernel Zea mays corn for at least 51 hours. The fermented whole kernel Zea mays corn has a final ethanol content F, wherein F is greater than E. More preferably, X is between 100 and 10,000. Even more preferably, E is less than 0.01% weight percent and F is greater than 0.10 weight percent.

Additionally, a method of producing a stabilized biomass is disclosed. An unmilled, biomass comprising water, active matter containing carbon atoms having an initial average oxidation state, and inactive matter is treated with a biological catalyst comprising a fermentation organism capable of consuming at least a portion of the active matter. The catalyst converts at least some of the active matter containing carbon atoms having an initial average oxidation state into a renewable material containing carbon atoms having an average oxidation state less than the initial average oxidation state of the active matter. A portion of the conversion of the active matter into the renewable material occurs substantially inside the biomass. Preferably, the biomass has a water content of between 18% and 50%. More preferably, the biomass has a water content of between 20% and 30%. Most preferably, the biomass has a water content of between 22% and 25%. The biological catalyst can be selected from the group consisting of cyanobacteria, fungus, algae, yeast, ethanologenic yeast, diatom and phytoplankton. The biomass can be selected from the group consisting of corn, rice, wheat, barley, rye, oats, sugar beets, sugar cane, Jerusalem artichoke and lignocellulose, and preferably is selected from the group consisting of whole corn, whole rice, whole wheat, whole barley, whole rye, whole oats, whole sugar beets, whole sugar cane and whole Jerusalem artichoke.

A preferred method of producing a biomass suitable for use in the production of renewable materials is disclosed. In this method, whole grain Zea mays corn comprising water, active matter containing carbon atoms having an initial average oxidation state, and inactive matter, is treated within 48 hours of harvest with a solution of Saccaromyces cerevisiae yeast in water, The solution of Saccaromyces cerevisiae yeast is capable of consuming at least a portion of the active matter containing carbon atoms having an initial average oxidation state and converting the active matter containing carbon atoms having an initial average oxidation state into a renewable material containing carbon atoms having an average oxidation state less than the initial average carbon oxidation state to form a biomass suitable for use in the production of renewable materials, Moreover, the whole grain Zea mays corn has not been force-dried.

The described embodiments are to be considered in all respects only as illustrative and not restrictive, and the scope of the invention is, therefore, indicated by the appended claims rather than by the foregoing description.

What is claimed is:

1. A biomass comprising:
active matter containing carbon atoms having an average oxidation state, inactive matter, a biological catalyst comprising a fermentation organism capable of converting the active matter into a renewable material and water;
the biomass having been processed in the absence of milling;
whereby the biomass is suitable for use in the production of renewable materials.

2. The biomass of claim 1, wherein the average oxidation state of the carbon atoms in the active matter is between −2 and −0.25.

3. The biomass of claim 1, wherein the biomass further comprises an antimicrobial agent.

4. The biomass of claim 1, the biological catalyst being selected from the group consisting of cyanobacteria, fungus, algae, yeast, ethanologenic yeast, diatom and phytoplankton.

5. The biomass of claim 1, the biological catalyst comprising ethanologenic yeast.

6. The biomass of claim 5, further having between 100 and 10,000 colony forming units of the biological catalyst per gram of biomass.

7. The biomass of claim 1, wherein the biomass has a moisture content of between 18% and 50% by weight.

8. The biomass of claim 1, wherein the biomass has a moisture content of between 20% and 30% by weight.

9. The biomass of claim 1, wherein the biomass has a moisture content of between 22% and 25% by weight.

10. The biomass of claim 1, wherein the biomass comprises a grain.

11. The biomass of claim 1, wherein the biomass comprises a whole grain.

12. The biomass of claim 1, the biomass being selected from the group consisting of sugar beets, sugar cane, Jerusalem artichoke and lignocellulose.

13. The biomass of claim 10, the grain being selected from the group consisting of corn, rice, wheat, barley, rye and oats.

14. The biomass of claim 11, the whole grain being selected from the group consisting of whole corn, whole rice, whole wheat, whole barley, whole rye and whole oats.

15. The biomass of claim 1, the biomass being selected from the group consisting of whole sugar beets, whole sugar cane and whole Jerusalem artichoke.

16. The biomass of claim 10, the grain being corn.

17. The biomass of claim 14, the biological catalyst comprising ethanologenic yeast.

18. The biomass of claim 17, further having between 100 and 10,000 colony forming units of the biological catalyst per gram of biomass.

19. A feedstock for yeast fermentation comprising unmilled, whole kernel Zea mays corn having an initial water content of between 22% and 25% and an initial ethanol content E;
the unmilled, whole kernel Zea mays corn having been inoculated with X colony forming units of Saccaromyces cerevisiae yeast per gram of unmilled, whole kernel Zea mays corn within 48 hours of harvest;
the whole kernel Zea mays corn further having been fermented by the at least X colony forming units of Saccaromyces cerevisiae yeast per gram of whole kernel Zea mays corn for at least 51 hours;
whereby the fermented whole kernel Zea mays corn has a final ethanol content F, and F is greater than E.

20. The feedstock of claim 19, wherein X is between 100 and 10,000.

21. The feedstock of claim 19, wherein E is less than 0.01 weight percent and F is greater than 0.10 weight percent.

22. A method of producing a stabilized biomass comprising:
treating an unmilled biomass comprising water, active matter containing carbon atoms having an initial average oxidation state, and inactive matter with a biological catalyst comprising a fermentation organism capable of consuming at least a portion of the active matter; and
converting at least a portion of the active matter containing carbon atoms having an initial average oxidation state into a renewable material containing carbon atoms having an average oxidation state less than the initial average oxidation state of the active matter;
wherein at least a portion of the conversion of the active matter into the renewable material occurs inside the biomass.

23. The method of claim 22, wherein the biomass has a water content of between 18% and 50%.

24. The method of claim 22, wherein the biomass has a water content of between 20% and 30%.

25. The method of claim 22, wherein the biomass has a water content of between 22% and 25%.

26. The method of claim 22, the biological catalyst being selected from the group consisting of cyanobacteria, fungus, algae, yeast, ethanologenic yeast, diatom and phytoplankton.

27. The method of claim 22, the biomass being selected from the group consisting of corn, rice, wheat, barley, rye, oats, sugar beets, sugar cane, Jerusalem artichoke and lignocellulose.

28. The method of claim 23, the biomass being selected from the group consisting of whole corn, whole rice, whole wheat, whole barley, whole rye, whole oats, whole sugar beets, whole sugar cane and whole Jerusalem artichoke.

29. A method of producing a biomass suitable for use in the production of renewable materials, comprising:
treating, within 48 hours of harvest, whole grain Zea mays corn comprising water, active matter containing carbon atoms having an initial average oxidation state, and inactive matter, with a solution of Saccaromyces cerevisiae yeast in water;
the solution of Saccaromyces cerevisiae yeast being capable of consuming at least a portion of the active matter containing carbon atoms having an initial average oxidation state and converting at least a portion of the active matter containing carbon atoms having an initial average oxidation state into a renewable material containing carbon atoms having an average oxidation state less than the initial average carbon oxidation state to form a biomass suitable for use in the production of renewable materials;
wherein the whole grain Zea mays corn has not been force-dried.
